(19) United States
(12) Patent Application Publication
(10) Pub. No.: US 2023/0159990 A1
(43) Pub. Date: May 25, 2023

(54) RESPONSIVE, CATALYTIC NUCLEIC NANOSTRUCTURES

(71) Applicants: National University of Singapore, Singapore (SG); Agency for Science, Technology and Research, Singapore (SG)

(72) Inventors: Huilin Shao, Singapore (SG); Yuan Chen, Singapore (SG); Noah Riandiputra Sundah, Singapore (SG); Rui Yuan Nicholas Ho, Proteos (SG)

(73) Assignees: National University of Singapore, Singapore (SG); Agency for Science, Technology and Research, Singapore (SG)

(21) Appl. No.: 17/917,826

(22) PCT Filed: Apr. 7, 2021
(86) PCT No.: PCT/SG2021/050194

§ 371 Date: Oct. 7, 2022

(30) Foreign Application Priority Data
Apr. 7, 2020 (SG) 10202003188S

(51) Int. Cl. C12Q 1/682; C12N 9/12

(52) U.S. Cl. CPC C12Q 1/682; C12N 9/1252; C12N 2310/128; C12N 2310/127; C12N 2310/122

(57) ABSTRACT

The present invention relates to signalling catalytic nucleic acid nanostructures that are responsive to polymerase activity, methods of their use, devices and kits comprising the same. More specifically, the present invention provides a catalytic signalling nanostructure comprising a DNAzyme/RNAzyme, such as G-quadruplex hemin, and a polymerase—responsive element. Polymerase elongation of the polymerase-responsive element eliminates catalytic activity of the DNAzyme/RNAzyme. The catalytic nucleic acid nanostructure can be used alone or paired with a target recognition nanostructure which can transduce molecular signals into polymerase activity, in an integrated circuit.

### FIELD OF THE INVENTION

The present invention relates to signalling catalytic nucleic acid nanostructures that are responsive to polymerase activity, methods of their use, devices and kits comprising same. More specifically, the present invention provides a sensitive catalytic signaling nanostructure comprising a DNAzyme/RNAzyme and a stimulus responsive element, which can be used alone or paired with a target recognition nanostructure which can transduce molecular signals into polymerase activity, in an integrated circuit.

### BACKGROUND OF THE INVENTION

Detection of nucleic acids has broad applications in, for example, diagnostics. Nucleic acid technologies have been increasingly adopted in clinical laboratories to provide unprecedented molecular information about infections [Niemz, A., Ferguson, T. M. & Boyle, D. S. Trends Biotechnol 29: 240-250 (2011); Nong, R. Y., et al., Expert Rev Proteomics 9: 21-32 (2012); Zumla, A. et al. Lancet Infect Dis 14: 1123-1135 (2014)].

Current detection of pathogen nucleic acids, is almost exclusively performed in large centralized clinical laboratories. This limited reach arises from the high complexity and cost associated with conventional technologies. Commercial assays leverage primarily on polymerase chain reaction (PCR) to amplify and detect specific DNA targets. Such systems not only necessitate bulky and specialized equipment for PCR thermal cycling and fluorescence measurements, but also require trained personnel to operate it. Advanced isothermal amplification assays have been developed to relieve the instrument needs for temperature cycling; nevertheless, these assays have their own limitations. For example, loop-mediated isothermal amplification (LAMP) has stringent sequence requirements and cannot be easily generalized [Zhao, Y., et al., Chem Rev 115: 12491-12545 (2015)]. Importantly, as with other nucleic acid amplification approaches, LAMP is prone to false-positives (e.g., from primer-dimer formation). Alternatively, sequence-specific signaling probes (e.g., fluorescent Taqman reporter) could be used to improve the detection accuracy; however, these probes are expensive and complex to implement [Gardner, S. N., et al., J Clin Microbiol 41: 2417-2427 (2003)]. As each piece of DNA target requires a dedicated, sequence-specific probe for coupled signaling during target amplification, the approach becomes increasingly costly and challenging to multiplex or perform complex computations [Juskowiak, B. Anal Bioanal Chem 399: 3157-3176 (2011)].

There is a need for an improved molecular platform to enable rapid, visual and modular detection of nucleic acids and other target molecules.

### SUMMARY OF THE INVENTION

The present invention is directed to responsive, catalytic nucleic acid nanostructures. These structures can be made responsive to polymerase activity and a variety of other stimuli and targets. The core of the structures comprises DNAzymes/RNAzymes, which are catalytic nucleic acids capable of performing specific chemical reactions, as well as different stimulus-responsive elements. As an example, the inventors designed and developed nanostructures to incorporate G-quadruplex Hemin DNAzyme and a polymerase-responsive element and demonstrated the structure's performance and compatibility to generate multi-modal readouts. By incorporating independent responsive elements, which can transduce molecular signals into polymerase activity, the system measures different molecular targets and/or combinations thereof, and demonstrates robust performance under different environment conditions. The present invention provides a new catalytic nanostructure for signaling, with improved sensitivity, speed to result, and robustness.

In a first aspect there is provided a catalytic nucleic acid nanostructure comprising a DNAzyme/RNAzyme and a stimulus responsive element. It would be understood that there are a number of known DNAzyme/RNAzymes that could be used for signaling in the present invention.

In some embodiments, the DNAzyme/RNAzyme may be selected from the group comprising:

a ribonuclease, such as ribonuclease 8-17, ribonuclease 10-23 or Dz10-66 deoxyribozyme;

a deoxyribonuclease, such as 10MD5 deoxyribozyme or 9NL27 deoxyribozyme;

a peroxidase, such as G-quadruplex Hemin;

an enzyme with ligation activity, such as E47 deoxyribozyme;

a phosphatase, such as 14VVM9 deoxyribozyme;

an amide hydrolyser, such as AmideAm1 deoxyribozyme; and

an RNA branching enzyme, such as 9F7 deoxyribozyme or 7S11 deoxyribozyme.

In a preferred embodiment the DNAzyme/RNAzyme is a G-quadruplex Hemin DNAzyme, Preferably, the G-quadruplex Hemin DNAzyme comprises the nucleotide sequence:

(SEQ ID NO: 1)
5′-CTGGGAGGGAGGGAGGGA-3′

In some embodiments, the stimulus responsive element comprises a polymerase-responsive element that inhibits the DNAzyme/RNAzyme activity in the presence of polymerase.

In some embodiments, the polymerase-responsive element lacks a hairpin structure. In a preferred embodiment the polymerase-responsive element comprises the nucleotide sequence set forth in SEQ ID NO: 2 and SEQ ID NO: 3:

(SEQ ID NO: 2)
5′-CTGGGAGGGAGGGAGGGAATGCTAACGCATTGTCGATAGC-3′
(SEQ ID NO: 3)
5′-GCTATCGACAATGCGTT-3′.

In some embodiments, the polymerase-responsive element has an internal hairpin structure.

In some embodiments, the polymerase-responsive element or self-priming portion of the signaling nanostructure comprises the nucleic acid sequence:

(SEQ ID NO: 10)
5′- AACGCATTGTCGATAGCTCAGCTGTCTGAGCTATCGACAATGCGT
T-3′.

In some embodiments, the catalytic nucleic acid nanostructure comprises a G-quadruplex Hemin DNAzyme and comprises a nucleic acid sequence selected from a group comprising:

(SEQ ID NO: 4)
5′-CTGGGAGGGAGGGAGGGAATGCTAACGCATTGTCGATAGCTCTGTCG
CTATCGACAATGCGTT-3′;
(SEQ ID NO: 5)
5′-CTGGGAGGGAGGGAGGGAATGCTAACGCATTGTCGATAGCTCTGTCG
CTATCGACAATGCGTTAGCAT-3′;
(SEQ ID NO: 6)
5′-CTGGGAGGGAGGGAGGGAATGCTAACGCATTGTCGATAGCTCTGTCG
CTATCGACAATGCGTTAGCATCCC-3′;
(SEQ ID NO: 7)
5′-CTGGGAGGGAGGGAGGGAATGCTAACGCATTGTCGATAGCTCTGTCG
CTATCGACAATGCGTTAGCATCCCTCCC-3′;
(SEQ ID NO: 8)
5′-CTGGGAGGGAGGGAGGGAATGCTAACGCATTGTCGATAGCTCTGTCG
CTATCGACAATGCGTTAGCATCCCTCCCTCCC-3′;
and
(SEQ ID NO: 9)
5′-CTGGGAGGGAGGGAGGGAATGCTAACGCATTGTCGATAGCTCTGTCG
CTATCGACAATGCGTTAGCATCCCTCCCTCCCTCCCAG-3′.

It would be understood that intermediate sequences within SEQ ID NO: 4 to 9 are intended to fall within the scope of the invention.

In some embodiments, when an internal hairpin structure is present polymerase elongation of the polymerase-responsive element eliminates catalytic activity.

In some embodiments, the DNAzyme/RNAzyme activity is a peroxidase activity.

In some embodiments, activity of a peroxidase substrate is detected by different modalities, including but not limited to colorimetric, fluorescence, electrochemical or luminescence means.

According to another aspect of the invention, there is provided a method of detecting polymerase activity in a test sample, comprising the steps of:

(a) providing a test sample;

(b) providing a composition comprising a catalytic nucleic acid nanostructure according to any aspect of the invention;

(c) contacting the sample in a) with the composition in b) in the presence of DNAzyme/RNAzyme substrate and, optionally, signal development reagents;

(d) detecting signal development, wherein the intensity of signal is inverse to the amount of polymerase activity in the sample.

In some embodiments, the test sample comprises a polymerase, such as DNA polymerase.

According to another aspect of the invention, there is provided a method of detecting target molecules in a sample, comprising the steps of:

(a) providing a test sample;

(b) providing a composition comprising at least one DNA polymerase enzyme and at least one recognition nanostructure, wherein the recognition nanostructure comprises a DNA polymerase enzyme-specific DNA aptamer adapted to recognize said target molecule in the sample

(c) providing a composition comprising at least one DNA polymerase enzyme and at least one recognition nanostructure, wherein the recognition nanostructure comprises a DNA polymerase enzyme-specific DNA aptamer having a conserved sequence region and a variable sequence region, wherein the variable sequence region comprises an overhang segment which is at least 10 nucleotides complementary to and forms a duplex with, a portion of the inverter oligonucleotide, wherein the inverter oligonucleotide is adapted to recognize said target molecule in the sample with a higher affinity than the variable duplex region;

(d) contacting the test sample with the composition of (b) or (c), wherein target molecule binding to:
(i) the recognition sequence region of the aptamer in (b) promotes the formation of a stable aptamer-DNA polymerase enzyme complex, thereby inhibiting DNA polymerase enzyme activity; or
(ii) the inverter oligonucleotide in (c) destabilizes the recognition nanostructure, thereby releasing the DNA polymerase enzyme from inhibition by the DNA aptamer;

(e) providing a catalytic nucleic acid nanostructure according to any aspect of the invention;

(f) contacting the nanostructure from step (b) or (c) in the presence of DNAzyme/RNAzyme substrate and, optionally, signal development reagents;

(g) detecting signal development, wherein the intensity of signal indicates;
(i) presence of target molecule in the sample when using composition (b); or
(ii) the absence of target molecule in the sample when using composition (c).

Suitable recognition nanostructures are described and defined in PCT Application Patent application PCT/SG2019/050328, published as WO 2020/009660, the contents of which are incorporated herein by reference.

In some embodiments, the DNA polymerase enzyme-specific DNA aptamer conserved sequence region of the recognition nanostructure comprises the nucleic acid sequence 5′-CAATGTACAGTATTG-3′ (SEQ ID NO: 18).

In some embodiments, the inverter oligonucleotide is at least one nucleotide longer than the aptamer duplex region. Preferably, the inverter oligonucleotide is about twice as long as the aptamer duplex region.

In some embodiments, about half of the inverter oligonucleotide length forms the aptamer-inverter duplex and about half forms an overhang segment.

In some embodiments, the method according to any aspect of the invention, further comprises providing a second recognition nanostructure complementary to a target nucleic acid different from the target nucleic acid of a first recognition nanostructure in the sample, for duplex detection.

In some embodiments, mismatches are introduced into the variable sequence region duplex to confer strong sequence specificity, useful for multiplex detection of closely related target nucleic acids such as for subtyping virus.

In some embodiments, the target is at least one nucleic acid selected from the group comprising DNA, RNA, PNA and other nucleic acid analogs.

In some embodiments, the target is at least one nucleic acid associated with a non-human or human disease, genetic variants, forensic, strain identification, environmental and/or food contamination.

In some embodiments, the target is a pathogen. In some embodiments the pathogen is a virus.

In some embodiments, the test sample comprises target molecules selected from a group comprising DNA, RNA, PNA, proteins, lipids, small molecules and metabolites and modifications thereof.

According to another aspect of the invention, there is provided a method of detecting target nucleic acids in a sample, comprising the steps of:

(a) providing a sample comprising nucleic acid;

(b) providing a composition comprising at least one DNA polymerase enzyme and at least one recognition nanostructure, wherein the recognition nanostructure comprises a DNA polymerase enzyme-specific DNA aptamer having a conserved sequence region and a variable sequence region, wherein the variable sequence region comprises an overhang segment which is at least 10 nucleotides complementary to a target nucleic acid in the sample; or

(c) providing a composition comprising at least one DNA polymerase enzyme and at least one recognition nanostructure, wherein the recognition nanostructure comprises a DNA polymerase enzyme-specific DNA aptamer and an inverter oligonucleotide, wherein the aptamer has a conserved sequence region and a variable sequence region, wherein the variable sequence region comprises an overhang segment which is at least 10 nucleotides complementary to, and forms a duplex with, a portion of the inverter oligonucleotide, wherein the inverter oligonucleotide is at least one nucleotide longer than the aptamer-inverter duplex and has more than 10 nucleotides complementary to a target nucleic acid in the sample;

(d) contacting the sample comprising nucleic acid with the composition of (b) or (c), wherein target nucleic acid binding to:
(i) the variable sequence region of the aptamer in (b) promotes the formation of a stable aptamer-DNA polymerase enzyme complex, thereby inhibiting DNA polymerase enzyme activity; or
(ii) the inverter oligonucleotide in (c) destabilizes the recognition nanostructure, thereby releasing the DNA polymerase enzyme from inhibition by the DNA aptamer;

(e) providing a catalytic nucleic acid nanostructure according to any aspect of the invention;

(f) contacting the nanostructure from step (d) in the presence of DNAzyme/RNAzyme substrate and, optionally, signal development reagents;

(g) detecting signal development, wherein the intensity of signal indicates;
(i) presence of target nucleic acid in the sample when using composition (b); or
(ii) the absence of target nucleic acid in the sample when using composition (c).

According to another aspect of the invention, there is provided a device comprising a catalytic nucleic acid nanostructure according to any aspect of the invention immobilized on a surface.

In some embodiments the device comprises:

(i) composition b) or composition c) comprising at least one DNA polymerase enzyme and at least one recognition nanostructure, as defined in claim 10, at a 1^{st} location;

(ii) catalytic nucleic acid nanostructure according to any aspect of the invention, attached at a 2^{nd} location; and

(iii) an intermediate stage for mixing of said detection nanostructures with sample nucleic acid to release active enzyme to said 2^{nd} location.

In some embodiments, the device is selected from a group comprising a microfluidic device and a lateral flow device.

In some embodiments, the device comprises an electrode.

According to another aspect of the invention, there is provided a nucleic acid detection kit comprising;

(a) a composition comprising at least one DNA polymerase enzyme and at least one recognition nanostructure, wherein the recognition nanostructure comprises a DNA polymerase enzyme-specific DNA aptamer having a conserved sequence region and a variable sequence region, wherein the variable sequence region comprises an overhang segment which is at least 10 nucleotides complementary to a target nucleic acid; and/or

(b) a composition comprising at least one DNA polymerase enzyme and at least one recognition nanostructure, wherein the recognition nanostructure comprises a DNA polymerase enzyme-specific DNA aptamer and an inverter oligonucleotide, wherein the aptamer has a conserved sequence region and a variable sequence region, wherein the variable sequence region comprises an overhang segment which is at least 10 nucleotides complementary to, and forms a duplex with, a portion of the inverter oligonucleotide, wherein the inverter oligonucleotide is at least one nucleotide longer than the aptamer-inverter duplex and has more than 10 nucleotides complementary to a target nucleic acid; optionally

(c) a catalytic nucleic acid nanostructure according to any aspect of the invention; optionally

(d) DNAzyme/RNAzyme substrate and, optionally,

(e) signal development reagents.

According to another aspect of the invention, there is provided a molecule detection kit comprising;

(a) a composition comprising at least one DNA polymerase enzyme and at least one recognition nanostructure, wherein the recognition nanostructure comprises a DNA polymerase enzyme-specific DNA aptamer having a conserved sequence region and a variable sequence region, wherein the variable sequence region comprises an overhang segment which is at least 10 nucleotides complementary to and forms a duplex with, a portion of the inverter oligonucleotide, wherein the inverter oligonucleotide is adapted to recognize said target molecule in the sample with a higher affinity than the variable duplex region;

(b) a catalytic nucleic acid nanostructure according to any aspect of the invention; optionally

(c) DNAzyme/RNAzyme substrate and, optionally,

(d) signal development reagents.

### BRIEF DESCRIPTION OF FIGURES

FIG. 1 shows the activity of DNAzyme nanostructures with different signaling elements. The activity of the DNAzyme nanostructures before incubation (a) and after 30 minutes incubation with DNA polymerase (b) or without (c) DNA polymerase. Experiments were done in triplicate and significance calculated using t-test with Bonferroni correction. n.s. means corrected p value >0.05, **<0.005, ****<0.00005

FIG. 2 shows the responsiveness of a DNAzyme signaling nanostructure after extension to a given nucleotide (SEQ ID NOs: 4 to 9). Experiments were done in triplicate.

FIG. 3 shows the types of readouts for a signaling nanostructure. The graphs indicate the signal produced by substrate with signaling nanostructure (black), and without signaling nanostructure (white). Type of readouts are further explained in Table 2. Electrochemistry measurements were performed with surface immobilized nanostructure, other readouts were performed with solution-based nanostructure. Experiments were done in triplicate.

FIGS. 4a and 4b show signaling nanostructure readout for different polymerase assay conditions. a) Time course of signal assay for different DNA polymerase dilutions (1×, 10×, 100×, and no polymerase). b) Signal from signaling nanostructure after incubation with DNA polymerase for 30 minutes when spiked with different amounts of contaminant (HCl) that inhibit enzyme activity. Experiments were done in triplicate.

FIG. 5 shows recognition nanostructure conditions for different substrates. Signaling nanostructure configurations for different targets. DNA/RNA (left and centre respectively) involve a target recognition element that base pairs and hybridizes to its complementary sequence. Protein, small molecule recognition involves an aptamer that folds to bind to its target (right). Graphs on bottom of schematic show signal from assay coupled to signaling nanostructure for different amounts of target. Experiments were done in triplicate.

FIGS. 6a and 6b show assay sensitivity and speed. a) Titration curve for assay coupled to signaling nanostructure for its specific target and a scrambled sequence. b) Time course experiment for 1010 specific and scrambled targets. Experiments were done in triplicate.

FIG. 7 shows that immobilized nanostructures preserve functionality. Non-immobilized recognition nanostructures (left) and surface immobilized recognition nanostructures (centre two, different configurations) are both able to preserve their specificity and sensitivity properties (Left, no target=no signal; Right, with target=high signal). The surface only control (right) does not have this property (high signal with or without target), showing that it is a property of the recognition nanostructure and not the surface. Experiments were done in triplicate.

### DETAILED DESCRIPTION OF THE INVENTION

Bibliographic references mentioned in the present specification are for convenience listed in the form of a list of references and added at the end of the Examples. The whole content of such bibliographic references is herein incorporated by reference.

### Definitions

Unless defined otherwise, all technical and scientific terms used herein have the same meaning as is commonly understood by one of skill in the art to which the invention belongs. Certain terms employed in the specification, examples and appended claims are collected here for convenience.

It must be noted that as used herein and in the appended claims, the singular forms “a,” “an,” and “the” include plural reference unless the context clearly dictates otherwise. Thus, for example, a reference to “a target sequence” includes a plurality of such target sequences, and a reference to “an enzyme” is a reference to one or more enzymes and equivalents thereof known to those skilled in the art, and so forth.

As used herein, the term “aptamer”, refers to single stranded DNA or RNA molecules.

An aptamer is capable of binding various molecules such as DNA, protein or small molecules with high affinity and specificity. For example, as used herein, in the absence of target DNA, protein or small molecules, the aptamer binds strongly with the polymerase to inhibit polymerase activity.

The phrases “nucleic acid” or “nucleic acid sequence,” as used herein, refer to an oligonucleotide, nucleotide, polynucleotide, or any fragment thereof, to DNA or RNA of genomic or synthetic origin which may be single-stranded or double-stranded and may represent the sense or the antisense strand, to peptide nucleic acid (PNA), or to any DNA-like or RNA-like material.

As used herein, the term “oligonucleotide”, refers to a nucleic acid sequence of at least about 6 nucleotides to 60 nucleotides, preferably about 15 to 30 nucleotides, and most preferably about 20 to 25 nucleotides, which can be used in PCR amplification or in a hybridization assay or microarray. As used herein, the term “oligonucleotide” is substantially equivalent to the terms “amplimers,” “primers,” “oligomers,” and “probes,” as these terms are commonly defined in the art. The recognition nanostructure may comprise an inverter oligonucleotide.

As used herein, the term “inverter sequence” or “inverter oligonucleotide” refers to an oligonucleotide which is complementary to a target nucleic acid sequence, of which a portion is involved in forming a duplex and a portion is involved in an overhang. Herein it is shown that a longer sequence of 20 nucleotides each for the duplex and overhang sequence robustly produces its inhibitory effect by stabilizing the aptamer binding to the DNA polymerase enzyme. This inhibitory effect can be removed in the presence of a complementary target at ambient temperatures. The presence/absence of the inverter sequence determines the functional state of the recognition element (e.g., on or off state). In its presence, the polymerase activity is turned on with targets; in its absence, the polymerase activity can be turned off with targets.

As used herein, the term “variable sequence region” refers to a region that determines the sequence specificity to the target sequences (i.e., defines the target sequences that can be recognized) on a recognition nanostructure. The inverter sequence and part of the aptamer sequence is contained within this variable sequence region. This region can be changed to enable detection of new targets. When no inverter is used the “variable sequence region” refers to an overhang segment on the aptamer that is complementary to target nucleic acid.

The term “sample,” as used herein, is used in its broadest sense. For example, a biological sample suspected of containing human papillomavirus (HPV) genome sequences, including but not exclusively HPV 6, 16, 18, 31, 33, and 5, may comprise a bodily fluid; an extract from a cell, chromosome, organelle, or membrane isolated from a cell; a cell; genomic DNA, RNA, or cDNA (in solution or bound to a solid support); a tissue; a tissue print; and the like.

It would be understood that oligonucleotides used in the present invention may be structurally and/or chemically modified to, for example, prolong their activity in samples potentially containing nucleases, during performance of methods of the invention, or to improve shelf-life in a kit. Thus, the aptamer and/or inverter and/or signaling nanostructure or any oligonucleotide primers or probes used according to the invention may be chemically modified. In some embodiments, said structural and/or chemical modifications include the addition of tags, such as fluorescent tags, radioactive tags, biotin, a 5′ tail, the addition of phosphorothioate (PS) bonds, 2′-O-Methyl modifications and/or phosphoramidite C3 Spacers during synthesis.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

### EXAMPLES

Standard molecular biology techniques known in the art and not specifically described were generally followed as described in Green and Sambrook, Molecular Cloning:

A Laboratory Manual, Cold Spring Harbour Laboratory, New York (2012).

### Example 1

### Design of Responsive, Catalytic Nucleic Nanostructures

The inventors designed and developed nucleic acid nanostructures to harbor catalytic activities; these catalytic activities are made responsive through the incorporation of stimulus-responsive elements. Specifically, we developed the core structure to comprise DNAzymes/RNAzymes, which are catalytic nucleic acids capable of performing specific chemical reactions [Li, W. et al., Nucleic Acids Research 44: 7373-7384 (2016)], and incorporated stimulus-responsive elements. In one example, we developed three-dimensional DNA structures to incorporate G-quadruplex Hemin DNAzyme (SEQ ID NO: 1), which has peroxidase activity.

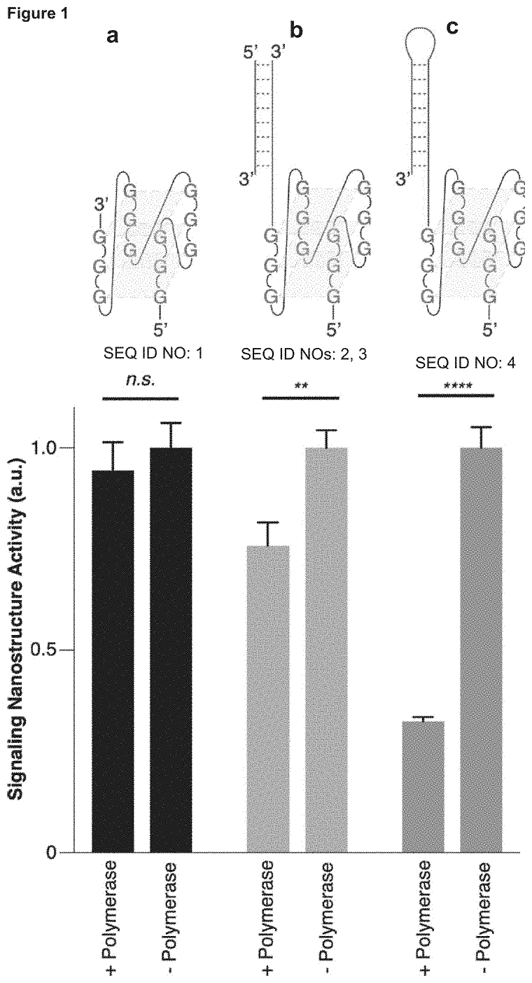

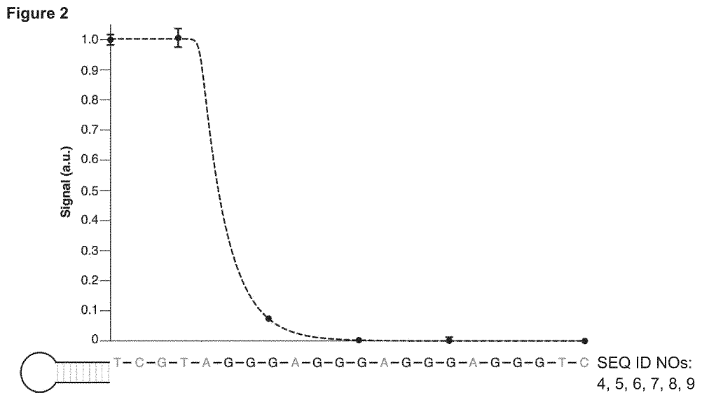

The inventors further designed and optimized the incorporation of a polymerase activity-responsive element to the nanostructures (SEQ ID NOs: 4 to 9). The created nanostructures thus contain both binding site for polymerase activity as well as intrinsic catalytic domains (SEQ ID NO: 2 and 3, SEQ ID NOs: 4 to 9), and are responsive to polymerase activity to change their catalytic peroxidase activity. Specifically, the responsive element provides a substrate for the polymerase activity, thereby unfolding and destroying the catalytic activity. While secondary and higher order DNA structures are known to inhibit polymerase activity [Nelms, B. L. and Labosky, P. A. Scientific Reports 1: 106 (2011)], we showed that, in the absence of the polymerase activity-responsive element, the designed nanostructure does not interfere with polymerase activity. After the incorporation of a polymerase activity-responsive element, in the absence of the polymerase activity, the catalytic activity of the nanostructure is preserved. In the presence of polymerase activity, the catalytic activity of the nanostructure is destroyed (FIG. 1). Of the polymerase-responsive elements tested, the best design had an internal hairpin structure, which we hypothesize can increase the stability of the polymerase responsive element through self-priming (FIG. 1) (SEQ ID No: 10). The inventors further optimized the design and position of the responsive element, with respect to the catalytic domain, by adjusting the length of the responsive element 5′ overhang to make the nanostructure highly responsive to polymerase activity. In the optimized design (SEQ ID NO: 4), polymerase elongation of a few bases was sufficient to completely destroy the catalytic activity; as any decrease in catalytic activity results in an exponential signal decrease, the designed nanostructure becomes highly sensitive to polymerase activity (FIG. 2). A list of nanostructure and target sequences is provided in Table 1.

TABLE 1
Nanostructure and target sequences

| | | SEQ ID |
|---|---|---|
| Description | Sequence | NO: |
| G-quadruplex | CTGGGAGGGAGGGAGGGA | 1 |
| Hemin | | |
| DNAzyme | | |
| Polymerase | CTGGGAGGGAGGGAGGGAATGCTAACGCATTGTCGATAGC | 2 |
| responsive | GCTATCGACAATGCGTT | 3 |
| element | | |
| G-quadruplex | CTGGGAGGGAGGGAGGGAATGCTAACGCATTGTCGATAGC | 4 |
| Hemin | TCTGTCGCTATCGACAATGCGTT | |
| DNAzyme | | |
| catalytic | | |
| nanostructure | | |
| 1 | | |
| G-quadruplex | CTGGGAGGGAGGGAGGGAATGCTAACGCATTGTCGATAGC | 5 |
| Hemin | TCTGTCGCTATCGACAATGCGTTAGCAT | |
| DNAzyme | | |
| catalytic | | |
| nanostructure | | |
| 2 | | |
| G-quadruplex | CTGGGAGGGAGGGAGGGAATGCTAACGCATTGTCGATAGC | 6 |
| Hemin | TCTGTCGCTATCGACAATGCGTTAGCATCCC | |
| DNAzyme | | |
| catalytic | | |
| nanostructure | | |
| 3 | | |
| G-quadruplex | CTGGGAGGGAGGGAGGGAATGCTAACGCATTGTCGATAGC | 7 |
| Hemin | TCTGTCGCTATCGACAATGCGTTAGCATCCCTCCC | |
| DNAzyme | | |
| catalytic | | |
| nanostructure | | |
| 4 | | |
| G-quadruplex | CTGGGAGGGAGGGAGGGAATGCTAACGCATTGTCGATAGC | 8 |
| Hemin | TCTGTCGCTATCGACAATGCGTTAGCATCCCTCCCTCCC | |
| DNAzyme | | |
| catalytic | | |
| nanostructure | | |
| 5 | | |
| G-quadruplex | CTGGGAGGGAGGGAGGGAATGCTAACGCATTGTCGATAGC | 9 |
| Hemin | TCTGTCGCTATCGACAATGCGTTAGCATCCCTCCCTCCCTC | |
| DNAzyme | CCAG | |
| catalytic | | |
| nanostructure | | |
| 6 | | |
| Self-priming | AACGCATTGTCGATAGCTCAGCTGTCTGAGCTATCGACAAT | 10 |
| portion of | GCGTT | |
| signaling | | |
| nanostructure | | |
| Recognition | TGCAAGGCCGGCTTCGCGGGCAATGTACAGTATTG | 11 |
| nanostructure | CCCGCGAAGCCGGCCTTGCACATGCCGGAGCCGTTGTCGA | 12 |
| Target nucleic | TCGACAACGGCTCCGGCATGTGCAAGGCCGGCTTCGCGG | 13 |
| acid 1 | G | |
| Target nucleic | UCGACAACGGCUCCGGCAUGUGCAAGGCCGGCUUCGCGG | 14 |
| acid 2 | G | |
| Scrambled | GAGTACCGCCCCCCGGTTTGGTACGGCGGCGGTGCGACA | 15 |
| sequence | A | |
| EPCAM | CAGACGCAACCTCTGTAGTGCAATGTACAGTATTG | 16 |
| aptamer/ | CACTACAGAGGTTGCGTCTGTCCCACGTTGTCATGGGGGG | 17 |
| inverter | TTGGCCTG | |
| DNA | CAATGTACAGTATTG | 18 |
| polymerase | | |
| aptamer | | |
| conserved | | |
| sequence | | |

### Example 2

### Multi-Modal Readouts of Catalytic Activities

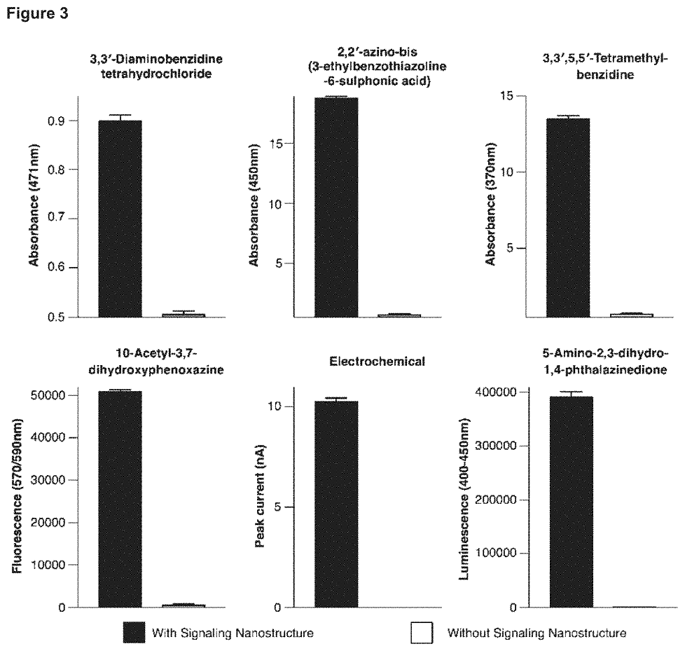

The catalytic activity of the nanostructure can be used as a signaling element, and assayed through various substrates, for rapid, ambient temperature detection. It can also be adapted for different readout modalities, including but not limited to colorimetric, fluorescence, electrochemical and luminescence (Table 2, FIG. 3).

TABLE 2
Types of readouts for signaling nanostructure

| Reagent | Type of readout |
|---|---|
| 3,3′-Diaminobenzidine tetrahydrochloride | Colorimetric |
| 2,2′-azino-bis(3-ethylbenzothiazoline-6-sulphonic acid) | Colorimetric |
| 3,3′,5′5′-Tetramethylbenzidine | Colorimetric |
| 10-Acetyl-3,7-dihydroxyphenoxazine | Fluorescence |
| — | Electrochemical |
| 5-Amino-2,3-dihydro-1,4-phthalazinedione | Luminescence |

Colorimetric readouts have the benefit of high portability. By immobilizing the nanostructure on an electrode, the system produces a highly sensitive, portable readout.

### Example 3

Detection of Polymerase Amount and/or Activity

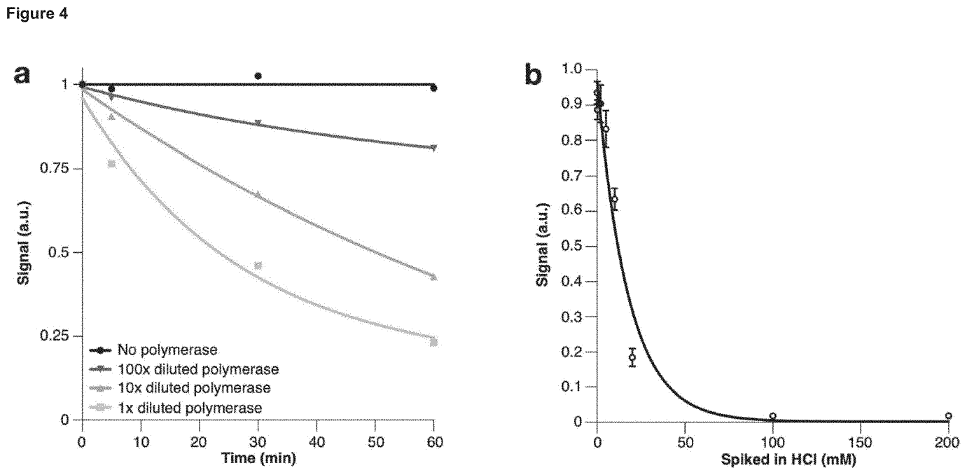

With the designed nanostructure (SEQ ID NO: 4), its catalytic activity can be used to directly measure polymerase amount. We mixed a solution containing the nanostructure with different amounts of polymerase, then took aliquots for measurement of DNAzyme activity at fixed timepoints. The reactions with less polymerase had a slower decay in DNAzyme activity over time as the lower amount of enzyme was unable to disrupt as much DNAzyme structure (FIG. 4a). This could be used to detect the amount of DNA polymerase, for example determining the yield of purified recombinant enzyme. The system can also be used to determine polymerase activity (activation or inhibition) (FIG. 4b), for example, in determining the effects of different amounts of chemical additives on the activity of a fixed amounts of enzymes.

### Example 4

### Detection of Different Stimuli

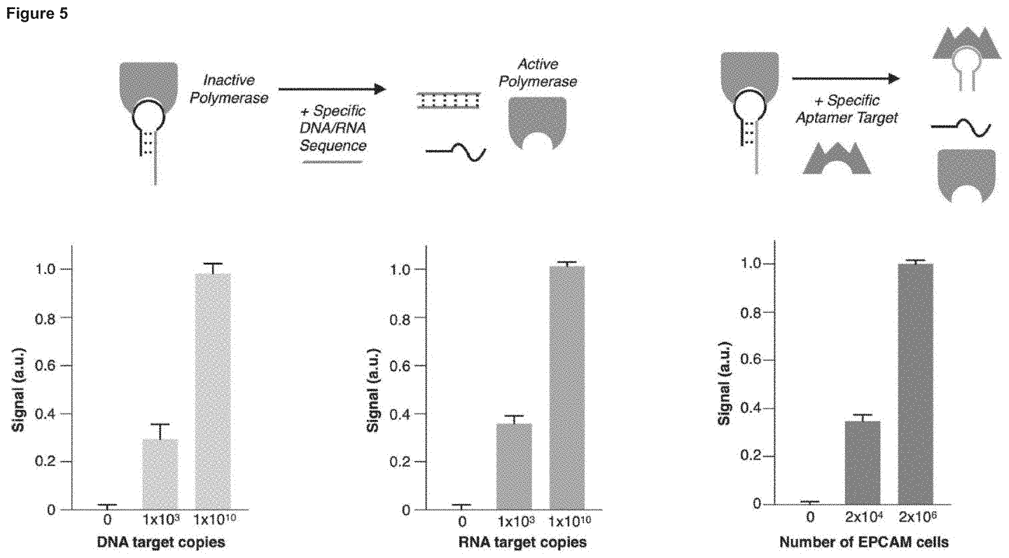

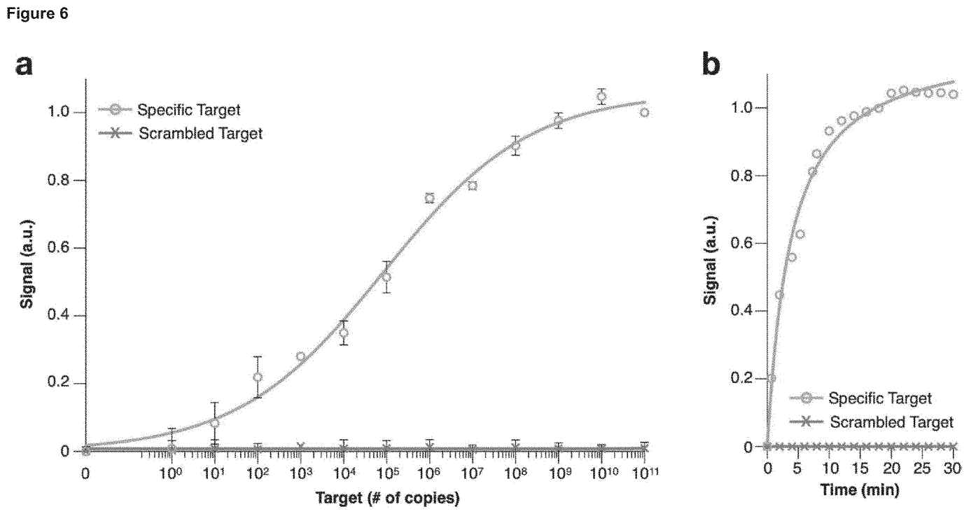

By coupling these catalytic nanostructures with other responsive nanostructures and other mechanisms that trigger DNA polymerase availability and/or activity, we can create a system that is responsive and specifically measures a varieties of other stimuli and target molecules, including but not limited to DNA, RNA, proteins, lipids, small molecules, metabolites and modifications, in a variety of different solution environments (e.g., cell lysates, chemical buffers). To demonstrate this, we used a specially designed recognition nanostructure (SEQ ID NOs: 11 and 12) that is responsive to a specific nucleic acid target (SEQ ID NOs: 13 and 14, Human Actin Beta, DNA and RNA, respectively) to activate/deactivate DNA polymerase [PCT Patent publication number WO 2020/009660 incorporated herein by reference; Ho, N. R. Y. et al., Nature Communications 9: 3238 (2018)], and coupled it upstream of the catalytic nucleic acid nanostructures. The integrated system could detect DNA and RNA targets with equal efficiency. By adapting the recognition nanostructure with aptamers against specific protein targets, this system can also be used to detect protein or small molecule targets. We designed a recognition nanostructure that is responsive to EPCAM protein by incorporating an aptamer for that protein into the inverter sequence (SEQ ID NOs: 16 and 17). As we added more cells which express the protein, we observed increasing amounts of polymerase activity (FIG. 5). By immobilizing the responsive DNAzyme nanostructure to the surface of an electrode, electrons produced from the DNAzyme activity could be rapidly shuttled to the electrode, thus producing a measurable electric current. This electrochemical readout enabled us to detect minute changes in DNAzyme activity. By coupling this with the recognition nanostructure, we were able to detect as little as 10¹ copies of specific target with no signal even with 10¹¹ copies of scrambled sequence (SEQ ID NO: 15) (FIG. 6a). The signal could be distinguished from scrambled target in a few minutes, and reach saturation in as little as 20 minutes (FIG. 6b).

### Example 5

### Immobilized Nanostructures for Functionality Control

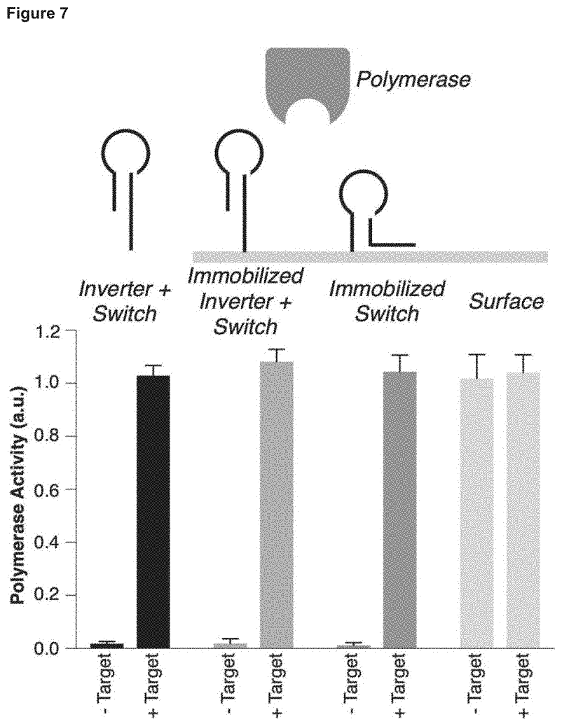

The different nanostructures could be surface immobilized, to control their functionality in different environments and sequential order of functionality. Specifically, the nanostructures described herein can be immobilized onto different surfaces such as gold, polystyrene, and silica can be used. These surfaces are functionalized through a variety of common bioconjugation reactions such as carbodiimide, succinimide, or dithiol crosslinking, gold-thiol, or avidin-biotin. Through the inclusion of linker and surface treatment groups (e.g., poly(ethylene glycol) or poly(ethylene oxide)), the surface density and molecular configurations could be optimized, to achieve functionality control and surface patterning. For example, using the immobilized recognition nanostructures described in Example 4 (SEQ ID NOs: 11 and 12) that recognize the gene for human beta actin, we demonstrate that the immobilization does not affect its function to bind and inhibit DNA polymerase. The same surface with no immobilized nanostructure was unable to inactivate the polymerase, demonstrating that it is a property of the recognition nanostructure. Importantly, the immobilized recognition nanostructure was able to bind to its programmed target nucleic acid sequence and activate the polymerase with comparable capacity (FIG. 7). The nanostructures can also be immobilized in different molecular configurations (FIG. 7), thereby enabling sequential order of functionality (information flow). Such immobilization enables array-type patterning for the detection of diverse targets [Yeh, E. C. et al., Sci Adv 3: e1501645 (2017)], and improves analytical performance in different environments that would otherwise inhibit various nanostructure functionalities (e.g., lysis buffer, detergents, ethylenediaminetetraacetic acid, or components of biological samples such as IgG, hemoglobin, proteases, and heparin) [Zumla, A. et al., Lancet Infect Dis 14: 1123-1135 (2014)]. Such systems thus enable direct detection of samples without requiring extensive purification steps.

### SUMMARY

Advantages of the present invention include:

1. Improved speed and sensitivity over non-catalytic nanostructures;
2. Improved performance with different readout modalities, in different environments, for enhanced portability;
3. Improved analytical performance in different environments that would otherwise inhibit different nanostructure functions;

### REFERENCES

1. Gardner, S. N., Kuczmarski, T. A., Vitalis, E. A. & Slezak, T. R. Limitations of TaqMan PCR for detecting divergent viral pathogens illustrated by hepatitis A, B, C, and E viruses and human immunodeficiency virus. J Clin Microbiol 41, 2417-2427 (2003).
2. Ho, N. R. Y. et al. Visual and modular detection of pathogen nucleic acids with enzyme—DNA molecular complexes. Nature Communications 9, 3238 (2018).
3. Juskowiak, B. Nucleic acid-based fluorescent probes and their analytical potential. Anal Bioanal Chem 399, 3157-3176 (2011).
4. Li, W. et al. Insight into G-quadruplex-hemin DNAzyme/RNAzyme: adjacent adenine as the intramolecular species for remarkable enhancement of enzymatic activity. Nucleic acids research 44, 7373-7384 (2016).
5. Niemz, A., Ferguson, T. M. & Boyle, D. S. Point-of-care nucleic acid testing for infectious diseases. Trends Biotechnol 29, 240-250 (2011).
6. Nelms, B. L. & Labosky, P. A. A predicted hairpin cluster correlates with barriers to PCR, sequencing and possibly BAC recombineering. Scientific Reports 1, 106 (2011).
7. Nong, R. Y., Gu, J., Darmanis, S., Kamali-Moghaddam, M. & Landegren, U. DNA-assisted protein detection technologies. Expert Rev Proteomics 9, 21-32 (2012).
8. Yeh, E. C. et al. Self-powered integrated microfluidic point-of-care low-cost enabling (SIMPLE) chip. Sci Adv 3, e1501645 (2017).
9. Zhao, Y., Chen, F., Li, Q., Wang, L. & Fan, C. Isothermal Amplification of Nucleic Acids. Chem Rev 115, 12491-12545 (2015).
10. Zumla, A. et al. Rapid point of care diagnostic tests for viral and bacterial respiratory tract infections—needs, advances, and future prospects. Lancet Infect Dis 14, 1123-1135 (2014).

1. A catalytic nucleic acid nanostructure comprising a DNAzyme/RNAzyme and a stimulus responsive element.

2. The catalytic nucleic acid nanostructure of claim 1, wherein the DNAzyme/RNAzyme is selected from the group comprising:
a ribonuclease;
a deoxyribonuclease;
a peroxidase;
an enzyme with ligation activity;
a phosphatase;
an amide hydrolyser; and
an RNA branching enzyme.

3. The catalytic nucleic acid nanostructure of claim 1, wherein the stimulus responsive element comprises a polymerase-responsive element that inhibits the DNAzyme/RNAzyme activity in the presence of polymerase.

4. The catalytic nucleic acid nanostructure of claim 3, wherein the polymerase-responsive element has an internal hairpin structure.

5. The catalytic nucleic acid nanostructure of claim 4, wherein polymerase elongation of the polymerase-responsive element eliminates catalytic activity.

6. The catalytic nucleic acid nanostructure of claim 1, wherein the DNAzyme/RNAzyme activity is a peroxidase activity.

7. The catalytic nucleic acid nanostructure of claim 6, wherein activity of a peroxidase substrate is detected by different modalities, including but not limited to colorimetric, fluorescence, electrochemical or luminescence means.

8. A method of detecting polymerase activity in a test sample, comprising the steps of:
(a) providing a test sample;
(b) providing a composition comprising a catalytic nucleic acid nanostructure of claim 3;
(c) contacting the sample in a) with the composition in b) in the presence of DNAzyme/RNAzyme substrate and, optionally, signal development reagents;
(d) detecting signal development, wherein the intensity of signal is inverse to the amount of polymerase activity in the sample.

9. A method of detecting target molecules in a sample, comprising the steps of:
(a) providing a test sample;
(b) providing a composition comprising at least one DNA polymerase enzyme and at least one recognition nanostructure, wherein the recognition nanostructure comprises a DNA polymerase enzyme-specific DNA aptamer adapted to recognize said target molecule in the sample;
(c) providing a composition comprising at least one DNA polymerase enzyme and at least one recognition nanostructure, wherein the recognition nanostructure comprises a DNA polymerase enzyme-specific DNA aptamer having a conserved sequence region and a variable sequence region, wherein the variable sequence region comprises an overhang segment which is at least 10 nucleotides complementary to and forms a duplex with, a portion of the inverter oligonucleotide, wherein the inverter oligonucleotide is adapted to recognize said target molecule in the sample with a higher affinity than the variable duplex region;
(d) contacting the test sample with the composition of (b) or (c), wherein target molecule binding to:
(i) the recognition sequence region of the aptamer in (b) promotes the formation of a stable aptamer-DNA polymerase enzyme complex, thereby inhibiting DNA polymerase enzyme activity; or
(ii) the inverter oligonucleotide in (c) destabilizes the recognition nanostructure, thereby releasing the DNA polymerase enzyme from inhibition by the DNA aptamer;
(e) providing a catalytic nucleic acid nanostructure of claim 3;
(f) contacting the nanostructure from step (b) or (c) in the presence of DNAzyme/RNAzyme substrate and, optionally, signal development reagents;
(g) detecting signal development, wherein the intensity of signal indicates;
(i) presence of target molecule in the sample when using composition (b); or
(ii) the absence of target molecule in the sample when using composition (c).

10. A method of detecting target nucleic acids in a sample, comprising the steps of:
(a) providing a sample comprising nucleic acid;
(b) providing a composition comprising at least one DNA polymerase enzyme and at least one recognition nanostructure, wherein the recognition nanostructure comprises a DNA polymerase enzyme-specific DNA aptamer having a conserved sequence region and a variable sequence region, wherein the variable sequence region comprises an overhang segment which is at least 10 nucleotides complementary to a target nucleic acid in the sample; or
(c) providing a composition comprising at least one DNA polymerase enzyme and at least one recognition nanostructure, wherein the recognition nanostructure comprises a DNA polymerase enzyme-specific DNA aptamer and an inverter oligonucleotide, wherein the aptamer has a conserved sequence region and a variable sequence region, wherein the variable sequence region comprises an overhang segment which is at least 10 nucleotides complementary to, and forms a duplex with, a portion of the inverter oligonucleotide, wherein the inverter oligonucleotide is at least one nucleotide longer than the aptamer-inverter duplex and has more than 10 nucleotides complementary to a target nucleic acid in the sample;
(d) contacting the sample comprising nucleic acid with the composition of (b) or (c), wherein target nucleic acid binding to:
(i) the variable sequence region of the aptamer in (b) promotes the formation of a stable aptamer-DNA polymerase enzyme complex, thereby inhibiting DNA polymerase enzyme activity; or
(ii) the inverter oligonucleotide in (c) destabilizes the recognition nanostructure, thereby releasing the DNA polymerase enzyme from inhibition by the DNA aptamer;
(e) providing a catalytic nucleic acid nanostructure of claim 3;
(f) contacting the nanostructure from step (d) in the presence of DNAzyme/RNAzyme substrate and, optionally, signal development reagents;
(g) detecting signal development, wherein the intensity of signal indicates;
(i) presence of target nucleic acid in the sample when using composition (b); or
(ii) the absence of target nucleic acid in the sample when using composition (c).

11. A device comprising a catalytic nucleic acid nanostructure of claim 1 immobilized on a surface.

12. The device of claim 11 comprising:
(i) composition b) or composition c) comprising at least one DNA polymerase enzyme and at least one recognition nanostructure, as defined in claim 10, at a first location;
(ii) catalytic nucleic acid nanostructure of claim 3, attached at a second location; and
(iii) an intermediate stage for mixing of said detection nanostructures with sample nucleic acid to release active enzyme to said second location.

13. The device of claim 11, selected from a group comprising a microfluidic device and a lateral flow device.

14. The device of claim 11, comprising an electrode.

15. A nucleic acid detection kit comprising;
(a) a composition comprising at least one DNA polymerase enzyme and at least one recognition nanostructure, wherein the recognition nanostructure comprises a DNA polymerase enzyme-specific DNA aptamer having a conserved sequence region and a variable sequence region, wherein the variable sequence region comprises an overhang segment which is at least 10 nucleotides complementary to a target nucleic acid; and/or
(b) a composition comprising at least one DNA polymerase enzyme and at least one recognition nanostructure, wherein the recognition nanostructure comprises a DNA polymerase enzyme-specific DNA aptamer and an inverter oligonucleotide, wherein the aptamer has a conserved sequence region and a variable sequence region, wherein the variable sequence region comprises an overhang segment which is at least 10 nucleotides complementary to, and forms a duplex with, a portion of the inverter oligonucleotide, wherein the inverter oligonucleotide is at least one nucleotide longer than the aptamer-inverter duplex and has more than 10 nucleotides complementary to a target nucleic acid; optionally
(c) a catalytic nucleic acid nanostructure of claim 3; optionally
(d) DNAzyme/RNAzyme substrate and, optionally,
(e) signal development reagents.

16. A molecule detection kit comprising;
(a) a composition comprising at least one DNA polymerase enzyme and at least one recognition nanostructure, wherein the recognition nanostructure comprises a DNA polymerase enzyme-specific DNA aptamer having a conserved sequence region and a variable sequence region, wherein the variable sequence region comprises an overhang segment which is at least 10 nucleotides complementary to and forms a duplex with, a portion of the inverter oligonucleotide, wherein the inverter oligonucleotide is adapted to recognize said target molecule in the sample with a higher affinity than the variable duplex region;
(b) a catalytic nucleic acid nanostructure of claim 3; optionally
(c) DNAzyme/RNAzyme substrate and, optionally,
(d) signal development reagents.

17. The catalytic nucleic acid nanostructure of claim 2, wherein the ribonuclease comprises ribonuclease 8-17, ribonuclease 10-23 or Dz10-66 deoxyribozyme;
the deoxyribonuclease comprises 10MD5 deoxyribozyme or 9NL27 deoxyribozyme;
the peroxidase comprises G-quadruplex Hemin;
the enzyme with ligation activity comprises E47 deoxyribozyme;
the phosphatase comprises 14WM9 deoxyribozyme;
the amide hydrolyser comprises AmideAm1 deoxyribozyme; and/or
the RNA branching enzyme comprises 9F7 deoxyribozyme or 7S11 deoxyribozyme.


SEQUENCE LISTING (18 sequences)

SEQ ID NO: 1 - DNA, 18 bp, Artificial Sequence
  G-quadruplex Hemin DNAzyme
ctgggagggagggaggga

SEQ ID NO: 2 - DNA, 40 bp, Artificial Sequence
  Polymerase-responsive element 1
ctgggagggagggagggaatgctaacgcattgtcgatagc

SEQ ID NO: 3 - DNA, 17 bp, Artificial Sequence
  Polymerase-responsive element 2
gctatcgacaatgcgtt

SEQ ID NO: 4 - DNA, 63 bp, Artificial Sequence
  G-quadruplex Hemin DNAzyme catalytic nanostructure 1
ctgggagggagggagggaatgctaacgcattgtcgatagctctgtcgctatcgacaatgcgtt

SEQ ID NO: 5 - DNA, 68 bp, Artificial Sequence
  G-quadruplex Hemin DNAzyme catalytic nanostructure 2
ctgggagggagggagggaatgctaacgcattgtcgatagctctgtcgctatcgacaatgcgttagcat

SEQ ID NO: 6 - DNA, 71 bp, Artificial Sequence
  G-quadruplex Hemin DNAzyme catalytic nanostructure 3
ctgggagggagggagggaatgctaacgcattgtcgatagctctgtcgctatcgacaatgcgttagcatccc

SEQ ID NO: 7 - DNA, 75 bp, Artificial Sequence
  G-quadruplex Hemin DNAzyme catalytic nanostructure 4
ctgggagggagggagggaatgctaacgcattgtcgatagctctgtcgctatcgacaatgcgttagcatccctccc

SEQ ID NO: 8 - DNA, 79 bp, Artificial Sequence
  G-quadruplex Hemin DNAzyme catalytic nanostructure 5
ctgggagggagggagggaatgctaacgcattgtcgatagctctgtcgctatcgacaatgcgttagcatccctccctccc

SEQ ID NO: 9 - DNA, 85 bp, Artificial Sequence
  G-quadruplex Hemin DNAzyme catalytic nanostructure 6
ctgggagggagggagggaatgctaacgcattgtcgatagctctgtcgctatcgacaatgcgttagcatccctccctccctcccag

SEQ ID NO: 10 - DNA, 46 bp, Artificial Sequence
  Self-priming portion of signaling nanostructure
aacgcattgtcgatagctcagctgtctgagctatcgacaatgcgtt

SEQ ID NO: 11 - DNA, 35 bp, Artificial Sequence
  Recognition nanostructure 1
tgcaaggccggcttcgcgggcaatgtacagtattg

SEQ ID NO: 12 - DNA, 40 bp, Artificial Sequence
  Recognition nanostructure 2
cccgcgaagccggccttgcacatgccggagccgttgtcga

SEQ ID NO: 13 - DNA, 40 bp, Artificial Sequence
  Target nucleic acid 1
tcgacaacggctccggcatgtgcaaggccggcttcgcggg

SEQ ID NO: 14 - RNA, 40 nt, Artificial Sequence
  Target nucleic acid 2
ucgacaacggcuccggcaugugcaaggccggcuucgcggg

SEQ ID NO: 15 - DNA, 40 bp, Artificial Sequence
  Scrambled sequence
gagtaccgccccccggtttggtacggcggcggtgcgacaa

SEQ ID NO: 16 - DNA, 35 bp, Artificial Sequence
  EPCAM aptamer/inverter
cagacgcaacctctgtagtgcaatgtacagtattg

SEQ ID NO: 17 - DNA, 48 bp, Artificial Sequence
  EPCAM aptamer/inverter 2
cactacagaggttgcgtctgtcccacgttgtcatggggggttggcctg

SEQ ID NO: 18 - DNA, 15 bp, Artificial Sequence
  Conserved sequence
caatgtacagtattg